(19) United States
(12) Patent Application Publication
(10) Pub. No.: US 2022/0273470 A1
(43) Pub. Date: Sep. 1, 2022

(54) ELECTRODE ASSEMBLY FOR A MYOELECTRIC PROSTHESIS

(71) Applicant: University of Salford, Salford (GB)

(72) Inventors: Ali Jabran, Manchester (GB); David Howard, Stockport (GB); John Head, Liverpool (GB); Laurence Kenney, Manchester (GB); Michael Prince, Stoke-on-Trent (GB)

(21) Appl. No.: 17/748,937

(22) Filed: May 19, 2022

Related U.S. Application Data
(63) Continuation of application No. PCT/GB2020/052966, filed on Nov. 20, 2020.

(30) Foreign Application Priority Data
Nov. 20, 2019 (GB) 1916904.4

(51) Int. Cl. A61F 2/72

(52) U.S. Cl. CPC A61F 2/72; A61B 5/296

(57) ABSTRACT

An electrode assembly for a myoelectric prosthesis allowing at least partial decoupling of the movement of the at least one electrode from the electrode housing case and from the prosthesis socket through the mounting of the at least one electrode using at least one biasing element. This allows the at least one electrode to be forced into contact with the patient in the target area using a moveable pressure application structure reducing/eliminating socket motion artifacts because the pressure is transferred toward the target area but the at least one electrode is not otherwise constrained to minimise or avoid the at least one electrode being dragged across the target area through the mounting to the prosthesis socket which causes socket motion artifacts.

### CROSS-REFERENCES TO RELATED APPLICATIONS

This application is a continuation of International Application No PCT/GB2020/052966, designating the United States, filed Nov. 20, 2020, which claims priority to GB1916904.4 filed on Nov. 20, 2019. The afore-mentioned applications are both incorporated by reference herein for all purposes.

### TECHNICAL FIELD

The present disclosure relates generally to electrode mounting assemblies for myoelectric prostheses and more particularly to an electrode assembly for improving the quality of the myoelectric signal by ensuring good electrode contact, good alignment with the muscle, and reduction or elimination of socket motion artefacts.

### BACKGROUND

Upper limb myoelectric prostheses are controlled by myoelectric signals produced by the muscles that remain within the residual limb. These signals are acquired via dry surface electrodes housed within the inner walls of the prosthetic interface or ‘socket’.

In myoelectric prostheses, the electrodes used to acquire the myoelectric signals require secure contact with the skin at all times for optimum function. Movement between the electrode and the surface of the residual limb can lead to the production of motion artifacts, or ‘false’ myoelectric signals, which can interfere with prosthesis control. Although the socket is rigid, it is not a perfect fit on the residual limb and the residual limb is fleshy and inherently mobile. This means that movement between the skin's surface and the socket wall, and hence the electrode, is distinctly possible.

In addition, once the electrode is secured within the socket wall, little if any adjustment or alteration to its contact security with respect to the surface of the skin or alignment with respect to the muscle fibres producing the myoelectric signal, is possible. The correct placement of the electrode within the socket, and its relative alignment and contact security with respect to the skin, is reliant upon the skill and experience of the Prosthetist.

The conventional solution to create a semi-rigid mounting for myoelectrical electrodes is to fix them into position using stiff rubber locating rods within a fixed housing created during the socket manufacturing process.

It is generally accepted that effective signal acquisition depends on the electrodes being securely attached to the skin's surface and also placed along the long axis of the target muscle fibres.

Anecdotal evidence suggests that standard clinical practice is to place the long axis of the electrodes parallel to the long axis of the residuum on an area with maximum signal strength which is typically achieved using ‘trial and error’. Providing an electrode site that will produce a clear, useable signal on the positive plaster model is also challenging, particularly for clinicians inexperienced in fitting myoelectric prostheses, and particularly in paediatric cases.

It would therefore be a significant advance in the field if an electrode assembly were provided that allowed for reduction in motion artifacts as a result of movement between the electrode and the surface of the residual limb and/or adjustability in any one or more of the electrode location, electrode orientation and skin-electrode contact security pressure.

### SUMMARY

According to a first aspect of the present disclosure, an electrode assembly for a myoelectric prosthesis is provided, the electrode assembly comprising:
a) An electrode housing case having at least one adjustable mounting portion to mount the electrode housing case relative to a prosthesis socket and thereby mount the electrode assembly relative to a target area on a residual limb;
b) At least one electrode mounted relative to the electrode housing case by at least one biasing element to bias the at least one electrode into a home position relative to the electrode housing case and spaced from the target area;
c) A pressure transfer unit to transfer pressure to the at least one electrode in a direction toward the target area; and
d) A moveable pressure application structure operatively associated with the pressure transfer unit, the moveable pressure application structure mounted relative to the electrode housing case and relative to the pressure transfer unit to apply adjustable pressure to the at least one electrode toward the target area and moveable between at least one lowered position in which the at least one electrode is in contact with the target area and at least one raised condition in which the at least one electrode returns to the home position.

This configuration allows at least partial decoupling of the movement of the at least one electrode from the electrode housing case and from the prosthesis socket through the mounting of the at least one electrode using the at least one biasing element. This allows the at least one electrode to be forced into contact with the patient in the target area using the moveable pressure application structure. The provision of the pressure transfer unit to transfer pressure applied using the moveable pressure application structure to the at least one electrode toward the target area reduces/eliminates socket motion artifacts because the pressure transfer unit transfers the pressure toward the target area but does not otherwise constrain the at least one electrode to minimise or avoid the at least one electrode being dragged across the target area through the mounting to the prosthesis socket which causes socket motion artifacts.

The pressure transfer unit will preferably transfer pressure to the at least one electrode in a direction toward the target area, but does not constrain the motion of the electrode relative to the housing case in any other way to avoid the socket dragging the electrode over the skin.

The adjustable electrode assembly for a myoelectric prosthesis of the present invention also advantageously provides an assembly allowing adjustability of one or more of the location and/or the orientation of the electrode housing case relative to the prosthetic socket and thereby the electrode relative to the residual limb. In an embodiment, the adjustable electrode assembly provides for adjustability of the skin-electrode contact security pressure to enhance the detection of the myoelectric signals produced by the muscles that remain within the residual limb to better control the myoelectric prostheses.

In a preferred form, the present embodiment further includes a pressure adjustment mechanism. The pressure adjustment mechanism preferably comprises an actuator mounted for user movement relative to the electrode housing case. The pressure adjustment mechanism typically further comprises a resiliently deformable adjustor mounted between the actuator and a portion of the moveable pressure application structure to adjust the pressure applied to the patient in the target area by the electrode.

In the present disclosure, the following axial system will be adhered to:
X axis is an axis parallel to the surface of the patient's skin in a first direction;
Y axis is an axis parallel to the surface of the patient's skin in a second direction orthogonal to the X axis; and
Z axis is an axis orthogonal to both the X axis and the Y axis, in a direction generally perpendicular to the surface of the patient's skin.

Typically, the pressure applied will be applied along the Z axis toward the surface of the patient's skin.

The electrode assembly of an embodiment has two main modes of operation, namely electrode up and electrode down as follows:

### a) Electrode Up Mode

In the electrode up mode, the pressure application structure is preferably in a raised condition so that the pressure transfer unit is no longer forcing the at least one electrode into contact with the target area. In this case, the at least one biasing element returns the at least one electrode to its home position within the electrode housing case. The at least one electrode will typically be lifted, by the at least one biasing element, away from the skin of the user. The prosthesis socket can then be doffed and donned.

Furthermore, in the electrode up mode, the at least one mounting portion typically allows the user to adjust the location (position and orientation) of the electrode housing case, and hence the at least one electrode, relative to the prosthesis socket and residual limb to enhance the detection of the myoelectric signals produced by the muscles that remain within the residual limb to better control the myoelectric prosthesis.

The at least one mounting portion will preferably allow small adjustments of the electrode housing case position in the X and Y directions and adjustment of its orientation around the Z-axis. Typically, the at least one mounting portion might provide for adjustment by allowing for the electrode housing to slide and/or rotate relative to the prosthesis socket. When located in a user selected location, the electrode housing case will normally be fixed in that location.

### b) Electrode Down Mode

In the electrode down mode, the moveable pressure application mechanism is preferably lowered such that the pressure transfer unit forces the at least one electrode against the skin of the user so that the at least one electrode can detect the myoelectric signals.

The pressure transfer unit preferably only applies pressure along the Z axis (toward the surface of the patient's skin) and allows free movement of the at least one electrode in the X and Y directions. Free rotation of the at least one electrode about all three axes (allowing five degrees of freedom of unrestrained movement) is also possible.

In this configuration and mode, small movements and rotations of the prosthesis socket relative to the skin will typically not lead to the at least one electrode being dragged over the skin by the prosthesis socket to at least reduce and preferably eliminate socket motion artifacts. In this mode, the at least one biasing element that locates the at least one electrode relative to the electrode housing case, is sufficiently compliant to allow the at least one electrode to move with the skin of the user.

The electrode housing case preferably includes a void surrounding the at least one electrode that provides for small movements and rotations of the prosthesis socket (and electrode housing case) relative to the skin (and the at least one electrode), thereby maintaining contact between the at least one electrode and the skin. The contact pressure can preferably be adjusted to maintain optimum contact pressure for detection of myoelectric signals.

In an embodiment, an electrode holder is provided to hold the electrode relative to the electrode housing case. In this embodiment, the at least one biasing element will normally be mounted to the electrode holder.

The electrode holder will preferably be held relative to the electrode housing case using one or more resiliently deformable biasing bodies to allow relatively small movements of the electrode holder, and thereby the electrode, relative to the electrode housing case and socket so that the electrode is not dragged over the skin causing socket motion artefacts.

Typically, a single electrode will be provided and a portion of the electrode will be in contact with the patient, typically the patient's skin in the target area, when the assembly is in a lowered use condition (electrode down mode). This will preferably allow the electrode to receive myoelectric signals produced by the muscles that remain within the residual limb in order to control the myoelectric prostheses.

When the moveable pressure application structure is in the raised position (electrode up mode), the pressure on the electrode will preferably be at least lessened. In a typical case, movement of the moveable pressure application structure to the raised position removes the pressure on the electrode entirely by separation of the pressure transfer unit, associated with the moveable pressure application structure, from the electrode and/or electrode holder.

Typically, the electrode housing case is attached/mounted to the prosthesis socket over a target area of the patient so that the electrode can contact the skin or body of the patient in the target area to receive signals from the target muscle fibres that control movement of the prosthesis. This normally occurs through a hole in the prosthetic socket.

In a preferred form, the electrode housing case is mounted relative to the prosthesis socket to allow for adjustment of the electrode housing case position and orientation relative to the socket and thereby relative to the patient's residual limb. The electrode housing case is preferably securable in position once the correct position and orientation is achieved.

Any mounting and securing mechanism that allows fixing of the position and/or orientation, at least temporarily but securely, can be used. For example, a track with a threaded member that can clamp the electrode housing case in position could be used, or a pin and slot sliding mechanism could be used. A clamping mechanism is preferred as it allows infinite adjustment over the length of the track.

Typically, more than one securing mechanism is provided. Preferably one securing mechanism is located on each of two opposed sides of the electrode housing case.

A preferred securing mechanism is the provision of an arcuate track opening in a flange extending laterally from a lower end of the electrode housing case. A threaded member preferably extends through the arcuate track opening. The threaded member is preferably associated with a clamping surface (which could be on a washer or similar) to abut an upper periphery of the arcuate track opening when tightened to allow the threaded member to clamp the electrode housing case in position. This configuration allows rotation of the electrode housing case about a substantially perpendicular axis (with reference to the skin surface) without allowing lateral movement.

One or more additional openings allowing an increased number of adjustable degrees of freedom can also be used, for example, one or more larger openings to allow three adjustable degrees of freedom—translation in X and Y directions and rotation about the Z-axis.

Preferably, the electrode housing case is formed from two housing portions, namely a hollow, lower housing portion to house the electrode holder and electrode and an upper housing portion to mount the moveable pressure application structure and the preferred pressure adjustment mechanism. The hollow, lower housing portion typically comprises the at least one mounting portion.

The lower housing portion is preferably rectangular in shape because it contains the typically rectangular electrode and its holder (although any shape could be used). The lower housing portion is also preferably larger than the upper housing portion.

The upper housing portion is preferably annular and elongate. In a typical embodiment of the invention, the upper housing portion takes the form of a tubular extension from an upper side of the lower housing portion.

An internal void is preferably provided within the lower housing portion. The void may be any shape. The void is preferably rectangular to assist with containment of the typically rectangular electrode holder, although usually of a larger dimension than the electrode holder allowing some movement of the electrode holder relative to the electrode housing case, thereby reducing or eliminating socket movement artefacts, but limiting the amount of the movement within the electrode housing case.

As mentioned above, a pair of mounting portions will preferably be provided on an outer part of the lower housing portion, typically provided on opposed sides of the lower housing portion. Each of the preferred pair of mounting portions is provided at a lower edge on opposed sides of the lower housing portion.

The upper housing portion is preferably integrally formed with the lower housing portion. As mentioned, the upper housing portion is preferably configured as a tubular portion with a bore extending therethrough. The bore typically extends from the top of the upper housing portion to communicate with the internal void in the lower housing portion.

The bore may be provided with a part of an adjustment mechanism depending on the configuration of the adjustment mechanism, for example the bore may be provided with an internally threaded portion.

The bore may also function as a guide for the resiliently deformable adjustor, for example by containing the resiliently deformable adjustor laterally, allowing only extension and compression.

A laterally extending shoulder may be provided between the lower housing portion and the smaller dimension, upper housing portion. The shoulder is typically provided as a part of a wall. The shoulder is preferably planar. At least one opening is preferably provided through the laterally extending shoulder. An opening is generally provided on two opposite sides of the housing case to allow at least one and preferably each of a pair of elongate arms of the moveable pressure application structure to pass through as explained further below.

A guide rebate is preferably provided into an outer surface of the upper housing portion. Preferably the guide rebate extends substantially vertically over the upper portion. Normally, a guide rebate is provided on at least two opposed sides of the upper housing portion.

A latching rebate is typically provided at an upper part of the or each guide rebate. The latching rebate will preferably extend circumferentially from the guide rebate. The latching rebate is typically provided in communication with the guide rebate such that rotation of the pressure application structure is normally required to latch the pressure application structure in the raised position (electrode up mode), and rotation in an opposite direction is required to release the pressure application structure from the latched position for movement towards the skin (electrode down mode).

The present embodiments may also comprise at least one electrode mounted relative to the electrode housing case and in contact with a patient in the target area.

Typically, a single electrode is provided and generally the electrode has a planar surface facing the patient.

The electrode is preferably provided in an electrode holder and the electrode holder is mounted relative to the electrode housing case.

The electrode holder may be biased into a raised condition within the electrode housing case. If so, the electrode holder is typically forced downwardly into contact with the patient by the moveable pressure application structure after the process of donning the prosthesis, returning to the raised position once the pressure is lessened or removed before the doffing process (or at any other time as desired).

The electrode holder is preferably mounted relative to the lower housing portion of the electrode housing case by one or more resiliently deformable bands or similar that allow small movements of the electrode holder (and electrode) within the electrode housing case (translation in the X and Y directions and rotation about the Z axis) but biases the electrode holder (and the electrode) into a home or reference position when the pressure is removed or lessened.

The resiliently deformable bands or similar preferably attach to the electrode holder and the electrode housing case. For example, the resiliently deformable bands or similar may extend through one or more guides on the electrode holder. Further the resiliently deformable bands or similar may extend through one or more openings provided in the electrode housing case.

The electrode holder preferably has at least one surface or portion to abut the pressure transfer unit when the movable pressure application structure is in the lowered position.

The present embodiments may also comprise a moveable pressure application structure which comprises a pressure transfer unit mounted relative to the electrode housing case and relative to the at least one electrode to apply adjustable pressure to the electrode in a direction of contact with the patient in the target area. The moveable pressure application structure is moveable between at least one lowered position in which the pressure is increased and at least one raised condition in which the pressure is lessened or removed.

The moveable pressure application structure is mounted relative to the electrode housing case, preferably allowing rotation of the moveable pressure application structure as well as linear movement of the moveable pressure application structure toward and away from the patient.

Preferably, the moveable pressure application structure is configured as an upper annular ring located outside the upper housing portion. A spaced apart lower annular receiver is also preferably provided to receive the pressure transfer unit located within the void in the lower housing portion. The upper annular ring and lower annular receiver are typically separated by at least one elongate arm. Typically, a pair of elongate arms are provided on the moveable pressure application structure to separate the upper annular ring and lower annular receiver. The lower annular receiver and the pressure transfer unit may be provided as a single part or as two or more parts.

Each of the elongate arms is preferably L-shaped having a longer portion and a shorter, perpendicular portion. In a preferred form, the longer portion extends from the upper annular ring of the moveable pressure application structure. The shorter, perpendicular portion preferably links a lower end of the longer arm to the lower annular receiver.

The inner side of the elongate arm and/or upper annular ring is preferably provided with a latching tongue. The latching tongue preferably extends parallel to the shoulder between the upper housing portion and the lower housing portion of the housing case. The latching tongue is typically dimensioned (height) to be received in or at least partially within the latching portion of the rebate on the outside of the upper housing portion. The latching tongue is typically dimensioned (length) to be received in or at least partially within the guide portion of the rebate on the outside of the upper housing portion. Provision of a latching tongue of these preferred dimensions allows the latching tongue to guide movement of the moveable pressure application structure and allow latching of the moveable pressure application structure in the raised position. The moveable pressure application structure can be latched in the raised position by rotation when the latching tongue is aligned with the latching portion of the rebate.

Preferably, the pressure transfer unit will be provided that applies pressure on the electrode in the Z direction (towards the skin) whilst allowing a relatively small amount of translation in the X and/or Y directions and also rotation about one or more of the X axis and/or Y axis and/or Z axis. Accordingly, a pressure transfer unit that is at least partially arcuate or has an at least partially arcuate portion is preferred.

The pressure transfer unit is preferably a unit provided at a lower end of the moveable pressure application structure, preferably within the electrode housing case.

The pressure transfer unit is typically a separate unit to the moveable pressure application structure and which is attached relative thereto. In one embodiment, the pressure transfer unit is a cylindrical shape with a lower, partially arcuate abutment portion associated therewith.

The abutment portion is typically a solid ball that can freely rotate within the pressure transfer unit assembly (i.e. a ball transfer unit is used). The abutment portion may take an alternative form such as for example, a closed bag or containment portion containing a fluid such as a liquid or gel. It is important that the abutment portion be configured to transmit pressure but also to allow that pressure to be transmitted effectively by allowing free translation and rotation or the electrode while transmitting pressure down on to the target area of a residual limb.

The present embodiments preferably further comprises a pressure adjustment mechanism. On one embodiment, the pressure adjustment mechanism comprises an actuator mounted for user movement relative to the electrode housing case and a resiliently deformable adjustor mounted between the actuator and a portion of the moveable pressure application structure, to adjust the pressure applied by the electrode to the patient, in the target area.

The actuator is normally provided at least partially within the bore in the upper portion of the housing case. A threaded engagement is preferably used to engage the actuator with the upper portion of the housing case, to allow a user to adjust the separation between the actuator and the pressure transfer unit, to in turn adjust the pressure on the pressure transfer unit, and thereby on the electrode.

The resiliently deformable adjustor is preferably a spring or similar. The preferred spring will be interposed between the actuator and the pressure transfer unit. In one embodiment, the spring is interposed between the actuator and upper part of the lower annular receiver.

The resilient adjustor is preferably confined within the bore of the upper housing portion to cause compression of the adjustor to change in a linear direction toward and away from the patient when the actuator is wound up and down to increase and decrease the pressure applied to the pressure transfer unit respectively.

After donning of the prosthesis, the pressure application structure is typically rotated out of the latching position and then moves downwardly, under the influence of the resilient adjuster, toward the patient's body to cause the electrode to abut the skin of the patient in the target area. In this electrode down mode, when the electrode is pressed against the skin by the pressure application structure, the pressure applied by the electrode onto the patient can be adjusted using the pressure adjustment mechanism by winding the actuator in the appropriate direction. The prosthesis is then ready for use and the assembly will preferably remain in this configuration until the electrode assembly is returned to the electrode up mode, when doffing can occur.

Before doffing of the prosthesis, the pressure application structure is lifted and rotated into the latching portion (electrode up mode). This will typically release the electrode which can then move back into the reference position if it has moved away from that position during use. The pressure adjustment mechanism may be relaxed prior to lifting the pressure application structure. In the electrode up mode, the position and/or orientation of the housing case can be adjusted as necessary using the mounting portions.

In an embodiment, the electrode assembly may further include a pressure application biasing assembly or mechanism to bias the electrode into the down position. The electrode may be movable against the bias, into the electrode up position.

At least a part of the pressure transfer unit may be mounted to or provided integrally with the electrode holder. For example, a partially hollow upstand may be provided extending upwardly from the electrode holder and topped by a ball transfer unit, in which a solid ball freely rotates within a portion of the upstand, provided at an upper end.

The abutment portion could be a solid ball that can freely rotate within a part of the pressure transfer unit assembly, but may take an alternative form such as for example a closed bag or containment portion containing a fluid such as a liquid or gel for example.

A pressure application biasing assembly or mechanism may be provided to bias the electrode into the down position and to bias the electrode into the home position when the electrode is moved to the electrode up position.

Two biasing assemblies or mechanisms can be provided, one pressure application biasing assembly or mechanism to bias the electrode into the down position and the aforementioned home position biasing assembly or mechanism to bias the electrode into the home position when the electrode is moved to the electrode up position.

The home position biasing assembly or mechanism that returns the electrode to its home position (in electrode up mode) should be sufficiently compliant so that the electrode is not dragged with the skin when lowered (in electrode down mode). Conversely, the pressure application biasing assembly mechanism typically maintains firm pressure with the skin while allowing the other 5 freedoms. For this reason, typically the home position biasing system returning the electrode to its home position should apply low forces in comparison to the pressure application biasing assembly of mechanism pushing the electrode against the skin.

The electrode assembly preferably provides an arrangement in which the movable pressure application structure is stabilised in the electrode up position and in the electrode down position (bistable), typically requiring forced movement from either one of these positions by the user.

In an embodiment, the electrode housing case may comprise a substantially planar plate mounted relative to the prosthesis socket. An adjustable mounting portion can be provided at two ends or sides of the substantially planar plate to mount the electrode assembly relative to a target area on a residual limb.

In this form, the electrode housing case will typically include a central opening to mount the electrode (or electrode holder) at least partially therein. The central opening will typically be larger than the electrode (or electrode holder) to provide clearance for the electrode (or electrode holder) to move within the opening.

An adjustable mounting arrangement including an adjustable mounting portion provided on the electrode housing case and a clamping member is typically provided at two ends or sides of the substantially planar plate to mount the electrode assembly relative to a target area on a residual limb. The clamping member is typically mounted relative to a stud or similar extending upwardly from the socket of the prosthetic. The clamping member may have an opening therein through which the stud or similar is received. The opening is typically larger than the stud. This will provide clearance between the stud and the periphery of the opening which in turn provides a user with the ability to adjust the position of the stud within the opening, thereby adjusting the position of the electrode housing assembly relative to the socket of the prosthetic.

The stud is typically provided with a fixing member to selectively fix the position of the clamping member. Normally, the stud is externally threaded and an internally threaded part is provided to fix the position of the clamping member when tightened. The clamping member and/or the mounting portion of the electrode housing case may have engagement formations, for example, serrations or similar to increase the security of the clamping between the parts. The engagement formations may be provided in an arc to allow the electrode housing case to move in an arc prior to being clamped. Up to 20° of arc may be provided.

The electrode housing case will typically be provided with at least one electrode mounting portion. Typically, the at least one electrode mounting portion will be provided adjacent to the opening through the electrode housing case. In an embodiment, a pair of opposed electrode mounting portions are provided. Each of the pair of opposed electrode mounting portions will normally be at an end of the opening, between the opening and the mounting portion. The electrode mounting portions will typically be elongate. The electrode mounting portions may extend across the electrode housing case.

The electrode mounting portions typically include structures to allow mounting of the electrode (or the electrode holder) relative to the opening of the electrode housing case. The structures may, in one form, include one or more arms relative to which the at least one biasing assembly or element of the home position biasing assembly or mechanism can be positioned to mount the electrode or the electrode holder. Typically, the at least one biasing assembly or element of the home position biasing assembly or mechanism will mount the electrode (or electrode holder) relative to the electrode housing case and bias the electrode (or electrode holder) into the home position in the electrode up position.

The electrode housing case may mount a number of arms of the movable pressure application structure. In an embodiment, the electrode mounting portions may mount a number of support arms of the movable pressure application structure.

In an embodiment, the movable pressure application structure may comprise a plate or similar mounted relative to the electrode housing case using a number of support arms. The support arms are typically pivotally or rotatably mounted relative to both the plate or similar and the electrode housing case. The plate may abut the pressure transfer unit.

Typically, the number of support arms provided to mount the plate or similar function to maintain the plate substantially parallel to the electrode housing case and so nominally, substantially parallel to the skin of the user. In an embodiment, the plate or similar will be rectangular and a support arm will be provided at or adjacent to each corner of the plate.

At least one of the support arms may be associated with a crank arm to be used to drive movement of the moveable plate between a raised position and a lowered position in which pressure is applied through the pressure transfer unit to the at least one electrode (or electrode housing).

In an embodiment, the crank arm may be provided at an angle to at least one of the support arms. The angle between the crank arm and the at least one support arm can be selected to exert a particular force versus displacement characteristic applied by the pressure application structure onto the pressure transfer unit. Normally the crank arm and the at least one support arm will be provided as a single member with two arm parts at an angle to each other, one support arm part mounting the preferred plate relative to the electrode housing case and a second arm part functioning as the crank arm.

The crank arm may be associated with the biasing means to bias the moveable pressure application structure downwardly. Any type of mechanism may be used to mount the biasing means. For example, am elongate biasing loop may be mounted relative to the crank arm and another part of the assembly and then to the crank arm to exert the downward force. Alternatively, a transverse member may be provided (between a pair of crank arms provided on either lateral side of the preferred plate) and associated with a torsion spring via a third arm to exert the downward force on the pressure application structure via the crank arm. In this configuration, a catch or similar may be provided to abut a portion of the mechanism to hold the preferred plate in the electrode up position.

The moveable pressure application structure may be locked in the up or down position. The moveable pressure application structure may be biased into either the up or down position by the use of a bi-stable arrangement (biased/stable into both the up and down position but less stable between these positions).

The home position biasing assembly or mechanism to bias the electrode or electrode holder into the home position may include one or more biasing elements. A single loop can be provided. More than one biasing element may be used to bias the electrode or electrode holder in different directions.

The pressure application biasing assembly or mechanism to bias the electrode into the down position may include one or more biasing elements. A single loop can be provided. More than one biasing element may be used to bias the electrode or electrode holder in different directions.

One or more biasing elements of difference resilience may be used to adjust the biasing force applied. This can be in relation to the biasing of the electrode or electrode holder into the home position and/or the biasing of the moveable pressure application structure into the up and/or down position.

In electrode up mode, the home position biasing assembly or mechanism returns the electrode to its home position. Conversely the pressure biasing assembly or mechanism may be bi-stable so that can be moved between the electrode up and electrode down modes and be biased into each of these modes.

The home position biasing assembly or mechanism may slacken as the pressure application plate lowers. In the electrode down position, the home position biasing assembly or mechanism may slacken so that in electrode down mode, the home position biasing assembly or mechanism has a lessened and preferably, no effect on the movement of the electrode (or electrode holder).

Providing more than one biasing element may allow adjustment but the provision of a single biasing element may balance the biasing force over multiple points of attachment.

The biasing elements may be elongate. A biasing element in the form or a compliant or resilient foam pad may be used.

The user may independently control the biasing arrangement to return the electrode or electrode housing to the home position, for example tensioning the biasing arrangement for electrode up position and releasing the biasing arrangement for electrode down position. This is typically provided in addition to a means of lifting or lowering the electrode.

The electrode assembly of the present invention therefore provides a simple and adjustable mechanism that provides partial decoupling of prosthetic socket and electrode, to reduce or eliminate socket movement artefacts, and also provides for adjustment of the pressure applied to an electromyographic electrode and the position and/or orientation of the electrode relative to the socket, and thereby the residual limb, to further reduce motion artefacts, improve electrode contact and allow better control of a prosthesis as a result.

According to a second aspect of the present invention, there is provided a prosthesis comprising at least one adjustable electrode assembly according to the first aspect of the invention.

The second aspect of the present invention may comprise any one or more of the features of the first aspect of the invention, as desired or appropriate.

### BRIEF DESCRIPTION OF THE DRAWINGS

In order that the invention may be more clearly understood one or more embodiments thereof will now be described, by way of example only, with reference to the accompanying drawings, of which:

FIG. 1 is an isometric view of an adjustable electrode assembly according to a preferred embodiment, mounted relative to a representation of the prosthesis socket.

FIG. 2 is a sectional view of the adjustable electrode assembly illustrated in FIG. 1 taken along line A-A.

FIG. 3 is an isometric view of the adjustable electrode assembly illustrated in FIG. 1 in the electrode down mode (electrode pressed against the skin).

FIG. 4 is an isometric view of the adjustable electrode assembly illustrated in FIG. 1 in the electrode up configuration (electrode raised for donning and doffing).

FIG. 5 is an isometric, partially exploded view of the electrode housing case and electrode holder of adjustable electrode assembly illustrated in FIG. 1.

FIG. 6 is a partially transparent, isometric view of the electrode housing case and electrode holder illustrated in FIG. 5 in an assembled, use configuration.

FIG. 7 is a view from below the configuration illustrated in FIG. 6.

FIG. 8 is a graphical illustration of motion artefacts represented as an electromyographic signal in mV acquired via an electrode in an upper limb myoelectric prosthesis in an ‘ideal secured to skin’ condition.

FIG. 9 is a graphical illustration of motion artefacts represented as an electromyographic signal in mV acquired via an electrode in an upper limb myoelectric prosthesis in a ‘conventional housing’.

FIG. 10 is a graphical illustration of motion artefacts represented as an electromyographic signal in mV acquired via an electrode in an upper limb myoelectric prosthesis using an adjustable electrode assembly according to a preferred embodiment of the present invention.

FIG. 11A is a side view of an assembly according to an embodiment in the electrode up configuration.

FIG. 11B is a side view of the assembly shown in FIG. 11A in the electrode down mode.

FIG. 11C is an isometric view of the configuration shown in FIG. 11A.

FIG. 12A is a side view of an assembly according to an embodiment in the electrode up configuration.

FIG. 12B is a top view of the assembly shown in FIG. 12A.

FIG. 13A is a top view of an assembly according to an embodiment.

FIG. 13B is a side view of the assembly shown in FIG. 13A.

FIG. 13C is a detailed isometric view from one side of an end of the assembly shown in FIG. 13A.

FIG. 13D is a detailed isometric view from the opposite side to that shown in FIG. 13C.

FIG. 14A is a side view of an assembly according to an embodiment in the electrode up configuration.

FIG. 14B is a side view of the assembly shown in FIG. 14A in the electrode down mode.

FIG. 14C is a top view of the assembly shown in FIG. 14A.

FIG. 14D is an isometric view of the assembly shown in FIG. 14A.

FIG. 15A is a side view of an assembly according to an embodiment in the electrode up configuration.

FIG. 15B is a side view of the assembly shown in FIG. 15A in the electrode down mode.

FIG. 15C is a top view of the assembly shown in FIG. 15A.

FIG. 16A is a side view of an assembly according to an embodiment in the electrode up configuration.

FIG. 16B is a top view of the assembly shown in FIG. 16A.

FIG. 17A is a side view of an assembly according to an embodiment in the electrode up configuration.

FIG. 17B is an isometric view of the assembly shown in FIG. 17A.

FIG. 18A is a side view of an assembly according to an embodiment in the electrode up configuration.

FIG. 18B is an isometric view of the assembly shown in FIG. 18A.

FIG. 19A is a side view of an assembly according to an embodiment in the electrode up configuration.

FIG. 19B is an isometric view of the assembly shown in FIG. 19A.

FIG. 20A is a side view of an assembly according to an embodiment in the electrode up configuration.

FIG. 20B is an isometric view of the assembly shown in FIG. 20A.

FIG. 21A is a side view of an assembly according to an embodiment in the electrode up configuration.

FIG. 21B is an isometric view of the assembly shown in FIG. 21A.

FIG. 21C is a top view of the assembly shown in FIG. 21A.

FIG. 21D is an isometric view from an end of the assembly shown in FIG. 21A.

FIG. 22A is a side view of an assembly according to an embodiment in the electrode up configuration with the lock engaged.

FIG. 22B is a side view of the assembly shown in FIG. 22A in the electrode down mode with the lock disengaged.

FIG. 22C is a side view of the assembly shown in FIG. 22B with the cord handle moved to the electrode down position.

FIG. 22D is a side view of the assembly shown in FIG. 22A in the electrode down mode.

FIG. 22E is a top view of the assembly shown in FIG. 22E.

FIG. 22F is an isometric view of the configuration shown in FIG. 22A.

FIG. 23A is a top view of an embodiment showing the mounting studs in the centred position relative to the mounting portions.

FIG. 23B is a top view of the configuration illustrated in FIG. 23A with the mounting studs out of centre relative to the mounting portions.

FIG. 23C is a top view of the configuration illustrated in FIG. 23B with the electrode housing in a rotated position relative to the mounting portions.

FIG. 23D is a detailed isometric view of the engagement between the electrode housing and the mounting portions in the embodiment illustrated in FIG. 23A.

### DETAILED DESCRIPTION

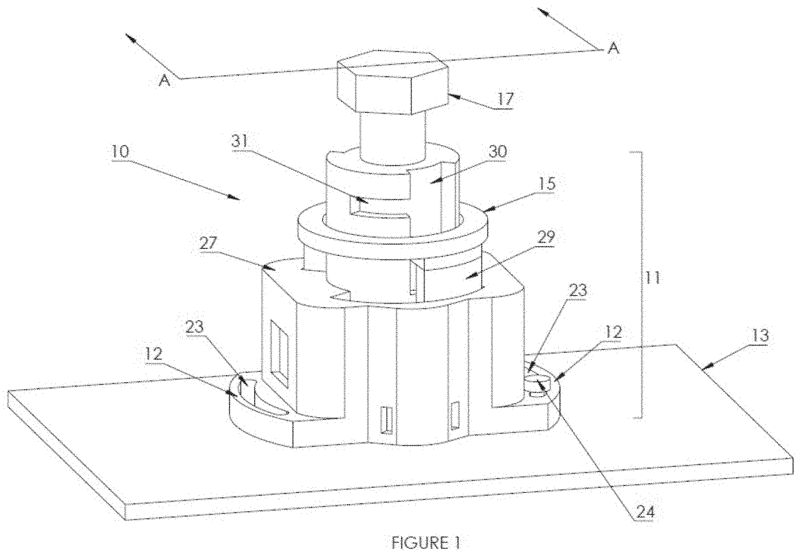

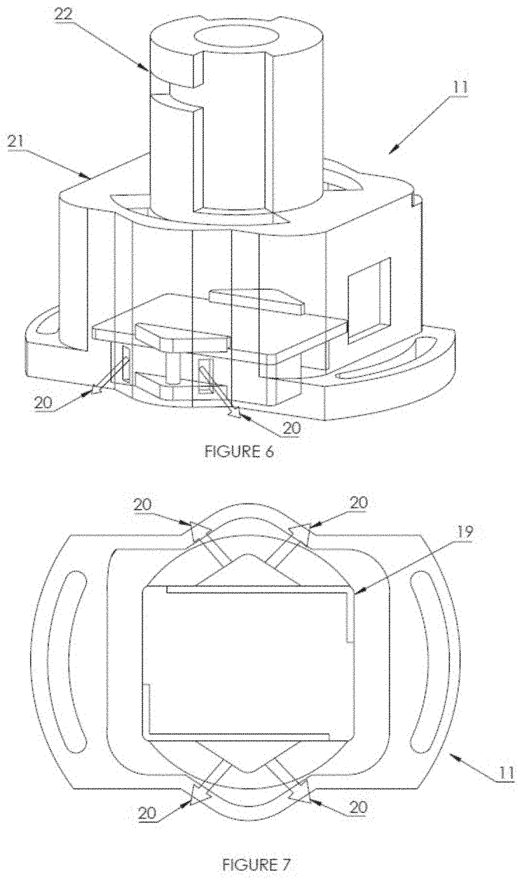

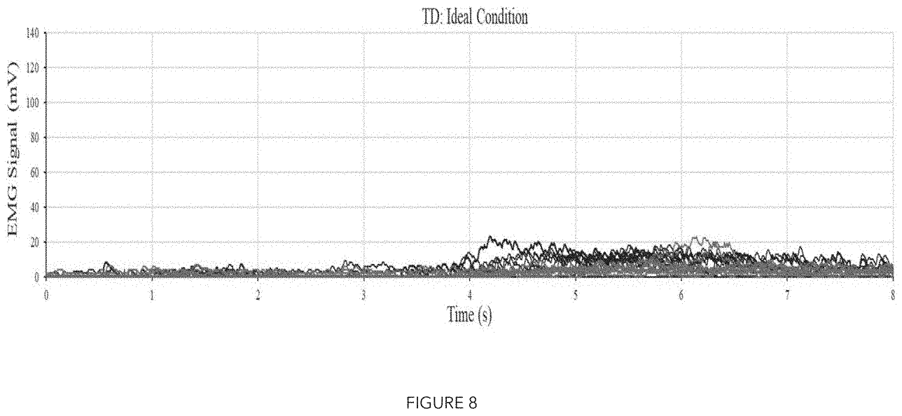

The adjustable electrode assembly 10 of the embodiments illustrated in FIGS. 1 to 7 comprises:
a) An electrode housing case 11 having a pair of mounting portions 12 to mount the electrode housing case 11 and thereby the adjustable electrode assembly 10 relative to the prosthesis socket 13 and thereby over a target area on a patient's residual limb;
b) An electrode 14 mounted relative to the electrode housing case 11 and in contact with the patient's body in the target area; and
c) A moveable pressure application wheel 15 operatively associated with a pressure transfer unit 16, the moveable pressure application wheel mounted relative to the electrode housing case 11 and relative to the electrode 14 to apply adjustable pressure to the electrode 14 in a direction of contact with the patient in the target area and moveable between at least one lowered condition (electrode down mode) in which the pressure on the patient's body in the target area is increased and at least one raised condition (electrode up mode) in which the pressure on the patient's body in the target area is lessened or removed.

In the illustrated form, a pressure adjustment mechanism is provided to act on the lower part of the moveable pressure application wheel 15 including a threaded actuator 17 mounted for user movement relative to the electrode housing case 11 and a resiliently deformable adjustor spring 18 mounted between the actuator 17 and a portion of the moveable pressure application wheel 15 to adjust the pressure applied by the electrode 14 to the patient's body in the target area.

Typically, the pressure applied will be applied along the Z axis toward the surface of the patient's skin allowing minor movement of the electrode in the five remaining degrees of freedom.

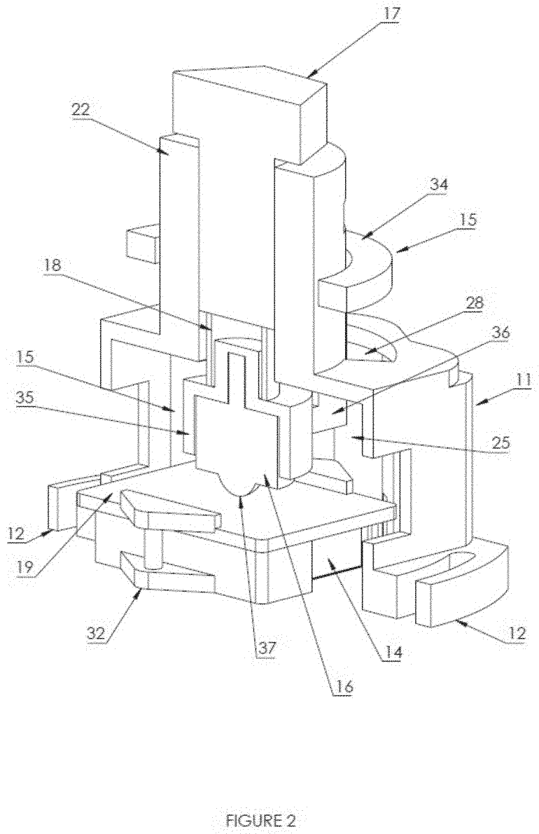

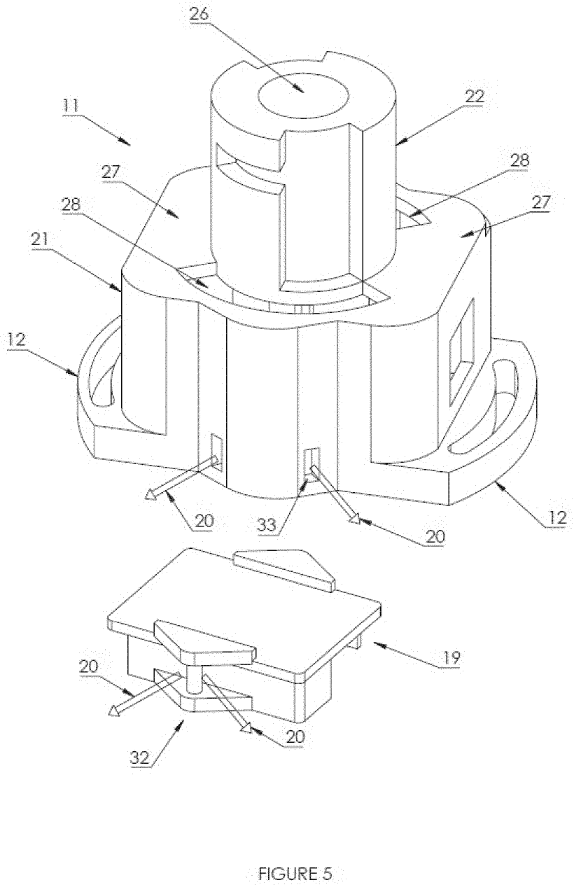

As shown in FIG. 2 in particular, an electrode holder 19 is provided to hold the electrode 14 relative to the electrode housing case 11. As shown in FIGS. 5 to 7, the electrode holder 19 of the illustrated embodiment is held relative to the electrode housing case 11 using one or more resiliently deformable biasing bands 20 to allow relatively small movements of the electrode holder 19, and thereby the electrode 14, relative to the electrode housing case and socket so that the electrode is not dragged over the skin causing socket motion artefacts.

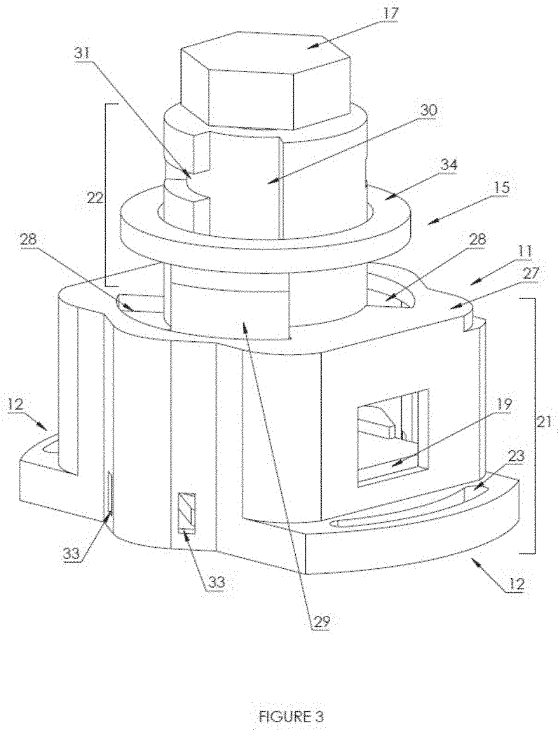

In use, a lower portion of the electrode 14 is in contact with the patient, typically the patient's skin in the target area, when the assembly 11 is in a lowered, use condition (illustrated in FIG. 3) to receive myoelectric signals produced by the muscles that remain within the residual limb in order to control the myoelectric prosthesis to which the assembly of the preferred embodiment is mounted.

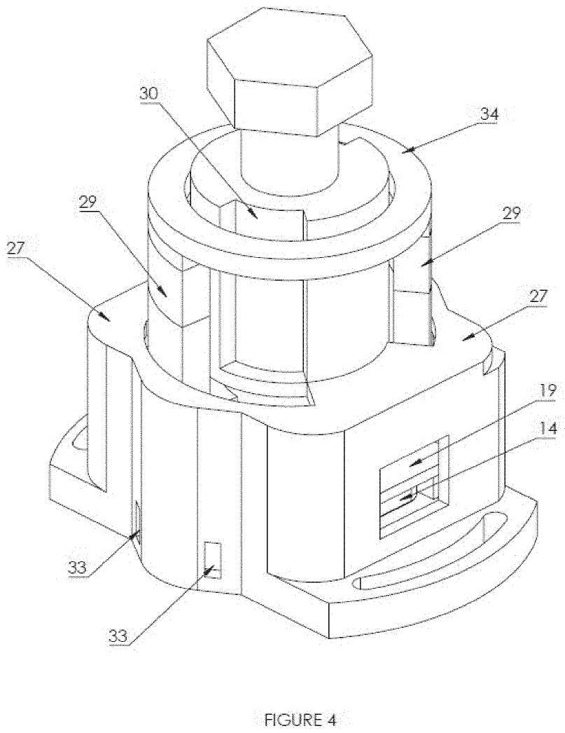

In the raised position (illustrated in FIG. 4), the pressure on the electrode 14 is lessened, and in most cases removed entirely by separation of the pressure transfer unit 16, associated with the moveable pressure application wheel 15, from the electrode 14 and/or electrode holder 19.

As shown, the electrode housing case 11 is attached/mounted to the prosthesis socket 13 over a target area of the patient' body so that the electrode 14 can contact the skin or body of the patient in the target area, through a hole in the prosthetic socket, to receive signals from the target muscle fibres that control movement of the prosthesis.

The electrode housing case 11 illustrated is mounted relative to the prosthesis socket to allow for adjustment of the orientation of the electrode housing case 11 relative to the socket and thereby relative to the patient's residual limb. The electrode housing case is securable in position once the correct orientation is achieved.

In the illustrated embodiment, each mounting portion 12 includes an arcuate track opening 23 provided in a flange extending laterally from a lower end of the electrode housing case with a threaded fastener 24 that can clamp the electrode housing case 11 in position as it allows infinite adjustment over the length of the track opening 23. As shown, a mounting portion and mechanism is located on each of two opposed sides of the electrode housing case 11. This configuration allows rotation of the electrode housing case about a substantially perpendicular axis (with reference to the skin surface) without allowing lateral movement.

The electrode housing case of the illustrated embodiment is formed from two housing portions, a lower housing portion 21 to house the electrode holder 19 and electrode 14 and which includes the mounting portions 12, and an upper housing portion 22 to mount the pressure adjustment mechanism.

The lower housing portion 21 illustrated is generally rectangular in shape and is also larger than the upper housing portion 22 which is annular and elongate, taking the form of a tubular extension from an upper side of the lower housing portion 21.

An internal void 25 as illustrated best in FIG. 2 is provided within the lower housing portion 21. The void 25 is generally rectangular in cross-sectional shape because it contains the typically rectangular electrode and electrode holder, although of a slightly larger dimension than the electrode holder allowing some movement of the electrode holder 19 relative to the electrode housing case 11, thereby reducing or eliminating socket movement artefacts, but limiting the amount of the movement within the electrode housing case 11.

The upper housing portion 22 of the illustrated embodiment is integrally formed with the lower housing portion 21. As mentioned, the upper housing portion 22 of the illustrated embodiment is configured as a tubular portion with a bore 26 therethrough which extends from the top of the upper housing portion 22 to communicate with the internal void 25 in the lower housing portion 21.

The bore 26 is provided with an internally threaded portion as part of the pressure adjustment to engage the actuator which has a correspondingly externally threaded portion.

As shown in FIG. 2, a lower portion of the bore 26 also functions as a guide for the resiliently deformable adjustor spring 18 by containing the resiliently deformable adjustor spring laterally, allowing only extension and compression.

A laterally extending shoulder wall 27 is provided between the lower housing portion 21 and the smaller dimension, upper housing portion 22. The shoulder wall 27 is planar with a pair of openings 28 provided through the laterally extending shoulder wall 27 to allow each of a pair of elongate arms 29 of the moveable pressure application wheel 15 to pass through as explained further below.

A guide rebate 30 with a latching rebate 31 is provided into an outer surface on two opposed sides of the upper housing portion 21. The guide rebate 30 extends substantially vertically over the upper housing portion 21.

The latching rebate 31 is provided at an upper part of each guide rebate 30 extending circumferentially from the guide rebate 30 but in communication with the guide rebate 30 as shown in FIG. 3 at least such that rotation of the pressure application wheel 15 can latch the pressure application wheel 15 in the raised position and rotation in an opposite direction is required to release the pressure application wheel 15 from the latched position, freeing it for movement toward and away from the patient.

As mentioned above, the electrode holder 19 of the illustrated embodiment is mounted relative to the lower housing portion 21 of the electrode housing case 11 by one or more resiliently deformable bands 20 to allow small movements of the electrode holder 19 (and electrode 14) within the electrode housing case 11 (translation in the X and Y directions and rotation about the Z axis) but which bias the electrode holder 19 (and the electrode 14) into a home or reference position when the pressure is removed or lessened.

The bands 20 of the illustrated embodiment attach to the electrode holder 19 and the electrode housing case 11 extending through one or more guides 32 on the electrode holder and through one or more openings 33 provided in the electrode housing case 11.

The electrode holder 19 has a flat upper surface which abuts the pressure transfer unit 16 when the movable pressure application wheel 15 is in the lowered position.

The moveable pressure application wheel 15 is mounted relative to the electrode housing case 11, allowing rotation of the moveable pressure application wheel 15 as well as linear movement of the moveable pressure application wheel 15 toward and away from the patient.

In the illustrated preferred embodiment, the moveable pressure application wheel 15 is configured as an upper annular ring 34 located outside the upper housing portion 22 and a spaced apart lower annular receiver 35 to receive the pressure transfer unit 16, located within the void 25 in the lower housing portion 21, separated by a pair of elongate arms 29. In the illustrated embodiment, the lower annular receiver 35 and the pressure transfer unit 16 are provided as two components.

Each of the elongate arms 29 as shown is L-shaped, having a longer upper portion and a lower, shorter, perpendicular portion 36. In the preferred form, the longer upper portion extends from the upper annular ring 34 of the moveable pressure application wheel 15 and the lower, shorter, perpendicular portion 36 links a lower end of the longer arm to the lower annular receiver 35.

The inner side of the elongate arm 29 and/or upper annular ring 34 of the illustrated embodiment is provided with a latching tongue (obscured in Figures). The latching tongue extends parallel to the shoulder wall 27 between the upper housing portion 22 and the lower housing portion 21 of the housing case 11. The latching tongue is dimensioned (height) to be received in or at least partially within the latching rebate 31 and is dimensioned (length) to be received in or at least partially within the guide rebate 30 to guide movement of the moveable pressure application wheel 15 up and down and allow latching in the raised position via rotation of the moveable pressure application wheel 15 when the latching tongue is aligned with the latching rebate 31.

The pressure transfer unit 16 applies pressure on the electrode in the Z direction (towards the skin) whilst allowing a relatively small amount of translation in the X and/or Y directions and also rotation about one or more of the X axis and/or Y axis and/or Z axis. Accordingly, in the preferred embodiment, the pressure transfer unit 16 has an at least partially arcuate abutment portion 37 to abut the electrode holder 19.

The pressure transfer unit is preferably a ball transfer unit, in which a solid ball freely rotates within the pressure transfer unit assembly, provided at a lower end of the moveable pressure application wheel 15, preferably within the electrode housing case.

In the illustrated embodiment, the pressure transfer unit 16 is a separate unit to the moveable pressure application wheel 15 and is attached relative thereto.

The abutment portion 37 of embodiment shown is a solid ball that can freely rotate within the pressure transfer unit assembly (i.e. a ball transfer unit is used), but may take an alternative form such as for example a closed bag or containment portion containing a fluid such as a liquid or gel for example.

The actuator 17 of the pressure adjustment mechanism is a bolt or similar with a portion provided at least partially within the bore 26 in the upper housing portion 22.

The resiliently deformable adjustor spring 18 is interposed between the actuator 17 and an upper part of the lower annular receiver 35. The adjustor spring 18 is partially confined within the bore 26 of the upper housing portion 22 to cause compression of the adjustor to change in a linear direction toward and away from the patient when the actuator 17 is wound up and down to increase and decrease the pressure applied to the pressure transfer unit 16 respectively.

The electrode down mode is best illustrated in FIG. 3. After donning of the prosthesis, the pressure application wheel 15 is typically rotated out of the latching position and then moves downwardly, under the influence of the resilient adjuster, toward the patient's body to cause the electrode 14 to abut the skin of the patient in the target area. In electrode down mode, when the electrode is pressed against the skin by the pressure application structure, the pressure applied by the electrode 14 onto the patient can be adjusted using the pressure adjustment mechanism by winding the actuator 17 in the appropriate direction.

The electrode up configuration is best illustrated in FIG. 4. Before doffing of the prosthesis, the pressure application wheel 15 is lifted and rotated into the latching rebate 31. This will typically release the electrode 14 which can then move back into the reference position if it has moved away from that position during use. The pressure adjustment mechanism may be relaxed prior to lifting the pressure application wheel 15. In electrode up mode, the orientation of the housing case 11 can be adjusted as necessary using the mounting portions 12.

The adjustable electrode assembly of the present invention therefore provides a simple and adjustable mechanism that provides partial decoupling of prosthetic socket and electrode, to reduce or eliminate socket movement artefacts, and also provides for adjustment of the pressure applied to an electromyographic electrode and the orientation of the electrode relative to the socket, and thereby the residual limb, to further reduce motion artefacts, improve electrode contact and allow better control of a prosthesis as a result.

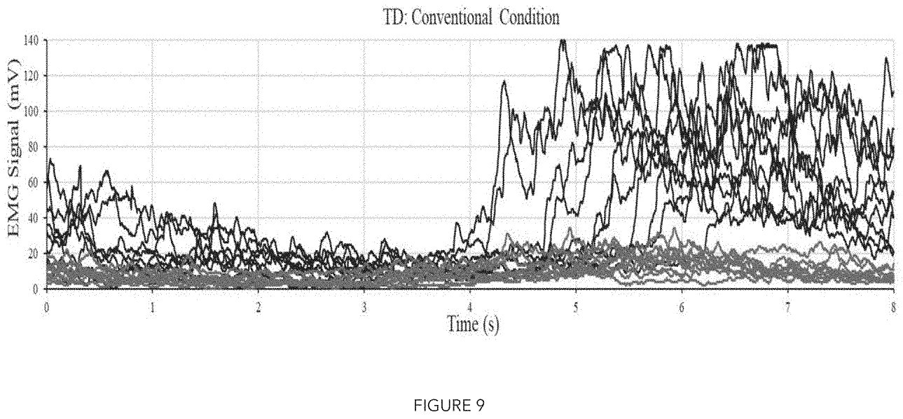

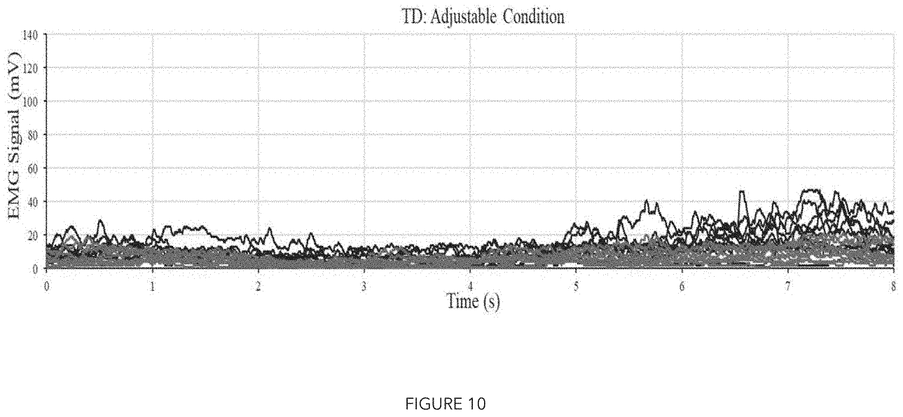

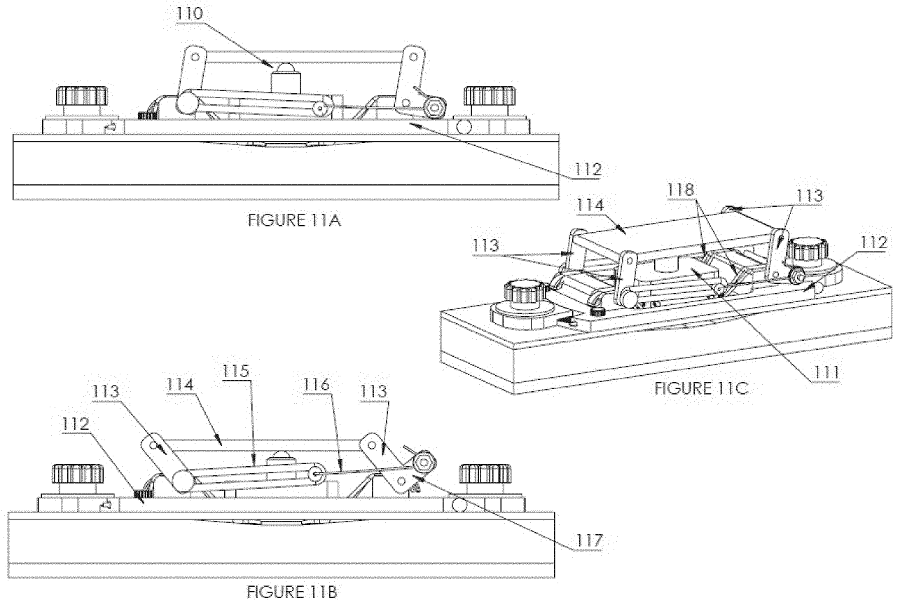

Example motion artefacts are illustrated in FIGS. 8 to 10 showing motion artefacts using three electrode conditions, namely an ‘ideal secured to skin’ condition (FIG. 8); a ‘conventional housing’ (FIG. 9); and using the adjustable housing of the preferred embodiment (FIG. 10). Of particular note is the reduction in motion artefacts when using the adjustable housing (in FIG. 10) versus those seen during use of the conventional housing (FIG. 9).

The effectiveness of the embodiments illustrated in FIGS. 1 to 7 relies, at least in part, on a biasing element being of low stiffness so that, in the electrode down mode, the electrode moves with the skin of the user, not with the socket. There is therefore, a trade-off between reliably returning the electrode to its home position in the electrode up mode and ensuring it moves with the skin in the electrode down mode.

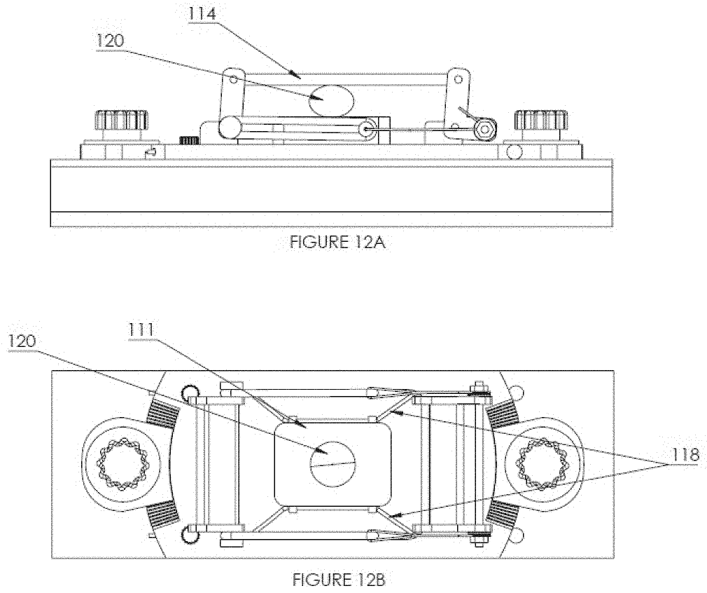

Further, the total height of the housing illustrated in FIGS. 1 to 7 (circa 50 mm) would not easily fit under clothing. FIGS. 11A to 23D show an assembly which has a lower profile than that illustrated in FIGS. 1 to 7. In many of the configurations illustrated in FIGS. 11A to 23D, a pair of resilient biasing arrangements are provided, a home return resilient biasing arrangement to bias the electrode (or the electrode holder into the home position and a pressure application resilient biasing arrangement to bias the moveable pressure application structure into the down (and/or up) position. The first resilient biasing arrangement and the second resilient biasing arrangement are preferably independent of one another.

FIGS. 11A to 11C show a low-profile design with the ball-unit integrated with the electrode. In the low-profile design illustrated in these Figures, the ball-unit 110, which provides the electrode (housed in the electrode holder 111) with 5 degrees of freedom of movement, is integrated with the electrode (or the electrode holder). This helps to apply the force centrally to the electrode.

The pressure application plate 114 is mounted relative to the electrode housing case 112 via a 4-arm mechanism 113 that ensures that the pressure application plate 114 remains parallel to the base of the housing case 112. The pressure application plate 114 is pulled down by the looped elastic element 115 shown in the Figures, which has adjustable pre-tension, in this case through the lengthening or slackening of the looped elastic element 115 using the bobbin attached to the arm 113 via member 116 via the crank arm 117. This is one example only: the elastic element could be any arrangement of elastic material or a conventional spring.

This arrangement shown forms a bi-stable mechanism, which provides a simple way of locking in the electrode up position. FIGS. 11A and 11B show the electrode up and electrode down positions respectively, illustrating the bi-stable action.

The angle between the bi-stable crank arm 117 and side-arm 113 supporting the plate 114 is shown as 90 degrees. This can be varied to produce different plate down-force versus displacement characteristics.

A home position biasing system consisting of elastic cords 118 returns the electrode to its home position when the pressure plate 114 is lifted. Alternative biasing arrangements are described below.

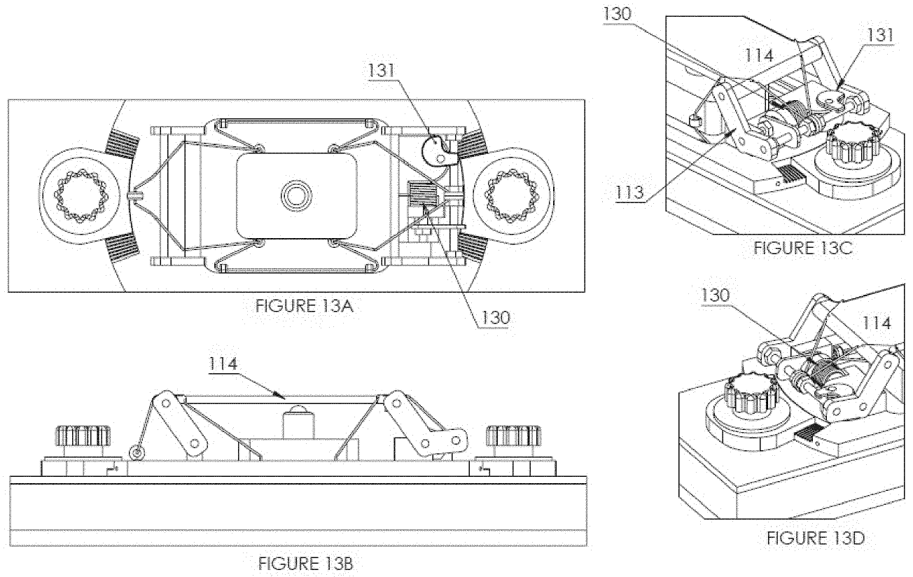

FIGS. 12A and 12B show a low-profile design similar to that illustrated in FIG. 11A to 11C with a flexible bag 120 replacing the ball-unit 110 (the plate 114 has been removed in FIG. 12B to show the structures beneath). The flexible fluid-filled bag 120 that provides the electrode with 5 degrees of freedom of movement when the electrode is pressed against the skin (electrode down mode). If the flexible bag 120 is attached to both the electrode holder 111 and the plate 114, in the electrode up mode, the biasing cords 118 only needs to centre the electrode as the bag 120 lifts it off the skin.

The elastic cords 118 return the electrode to its home position when the pressure plate 114 is lifted, in a similar way to the embodiment illustrated in FIGS. 11A to 11C. Alternative biasing arrangements are described below.

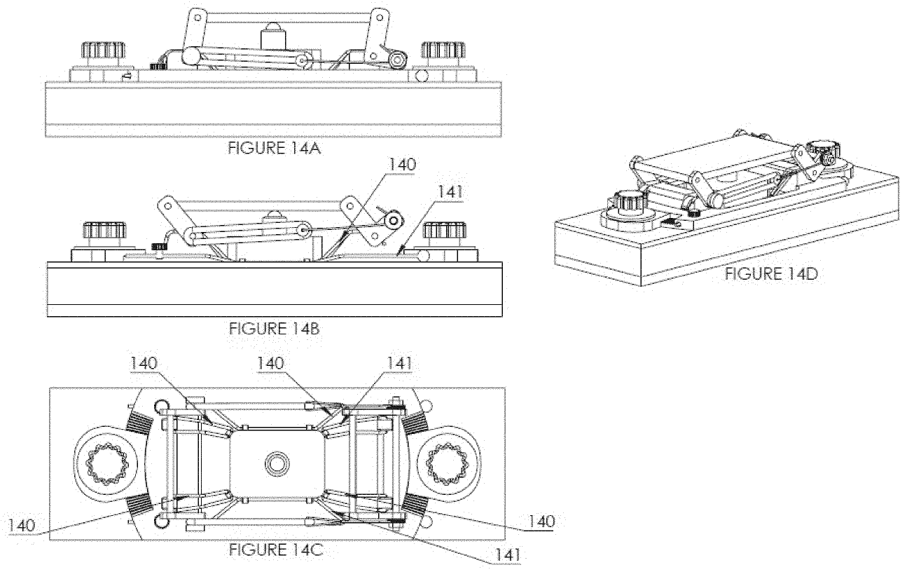

FIGS. 13A to 13D show a low-profile design with the looped elastic element of FIGS. 11A to 12B replaced by a torsion spring 130. An adjustable torsion spring 130 is an example of an alternative way of spring loading the arms 113 (albeit indirectly) so that the plate 114 pushes the electrode against the skin. This configuration lacks the advantage of being bi-stable and, hence, a catch 131 is provided to hold the plate 114 in the up position, as illustrated in FIGS. 13C and 13D. Alternative biasing arrangements are described below.

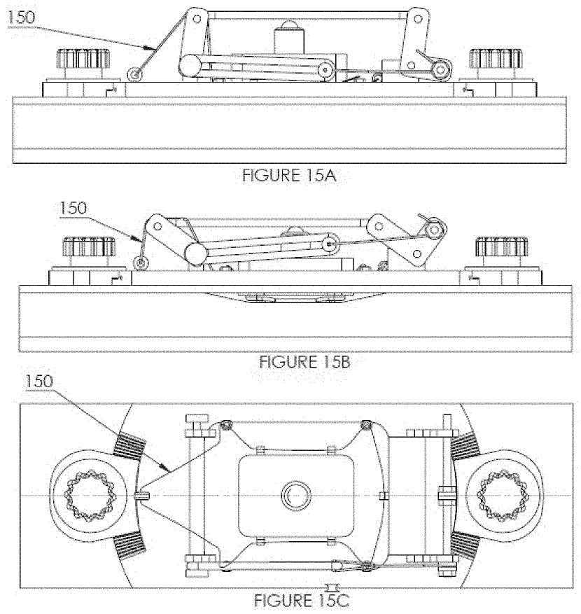

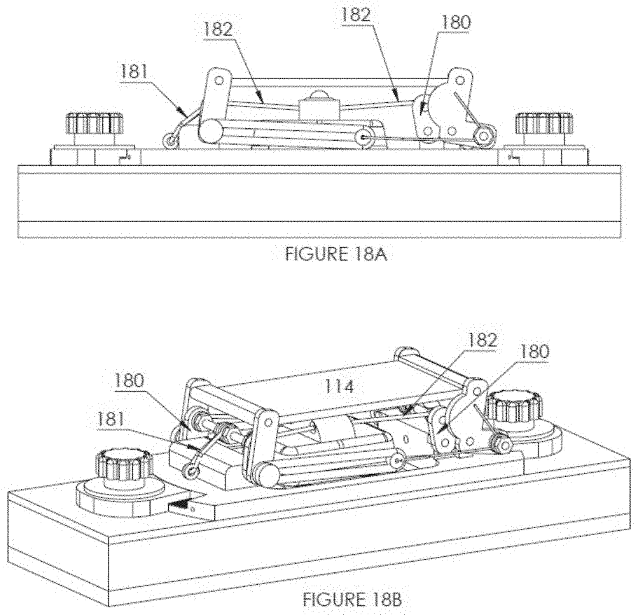

Alternative biasing arrangements to those illustrated in FIGS. 11A to 13D could be used to return the electrode to its home position when in the electrode up mode. The biasing arrangements include one or more of using fixed elastic bias cords, a means of slackening the cord as the pressure application plate descends, routing the bias cords via the electrode case (as shown in FIGS. 14A to 14D) and/or the pressure application plate (as shown in FIGS. 13A to 13D), foam or similar (as shown in FIGS. 17A and 17B) to centralise the electrode as it is returned to its home position, sprung arms to tension stiff biasing cords and a means for the user to independently control the biasing cords.

FIGS. 14A to 14D illustrate the use of fixed vertical elastic bias cords 140 and lateral elastic bias cords 141. This arrangement of elastic biasing cords is similar to the configuration illustrated in FIGS. 11A to 11C but the ball-unit 110 remains in contact with the plate 114 when it is lifted off the skin. This configuration also has improved geometry for centralising the electrode in its home position.

The geometry of the elastic biasing cords could be chosen to ensure that the ball-unit 110 is not in contact with the plate 114 when it is lifted off the skin. In the electrode up mode, the ball may be either held against the plate or there may be a gap between the ball and plate.

It should also be noted that the elastic biasing cords could be replaced by conventional springs.

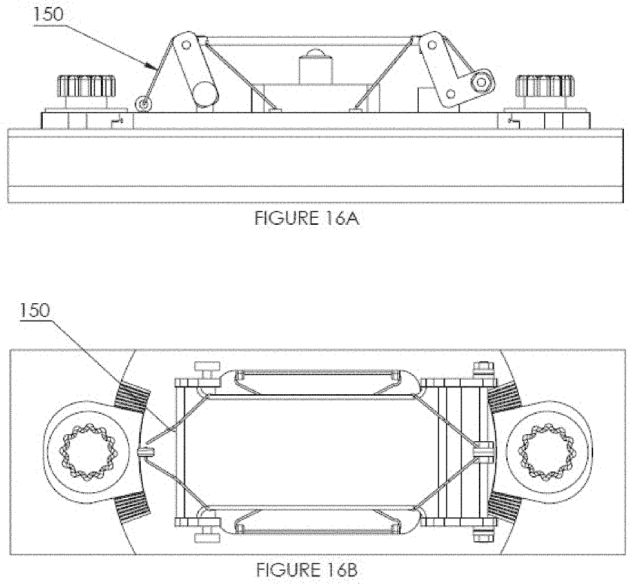

FIGS. 15A to 15C show a single loop of elastic bias cord 150 that slackens as the electrode is lowered onto the skin from the position in FIG. 15A to that shown in FIG. 15B. This arrangement uses a single loop of elastic bias cord 150, which slackens when the electrode is pressed downwardly against the skin. This happens because the segments of cord on the left (from plate to left most guide) shorten as the plate 114 moves down. This overcomes the trade-off between reliably returning the electrode to its home position in the electrode up mode and ensuring it moves with the skin in the electrode down mode mentioned above.

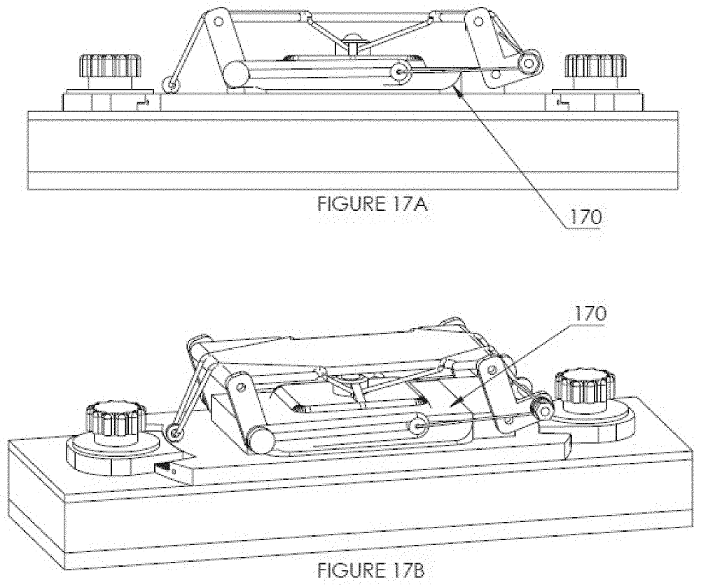

In the embodiment illustrated in FIGS. 16A and 16B (plate removed for clarity), the single loop of elastic bias cord 150 passes via the plate 114 to lift the electrode as the plate 114. This embodiment is similar to the arrangement of FIGS. 15A to 15C, except the path followed by the cord 150 also passes through guides on the moving plate 114. This means that, as the plate 114 is lifted, the electrode is also lifted. This may provide better biasing in both vertical and lateral directions and thereby reduce the sensitivity of the home location to socket orientation.

In the embodiment illustrated in FIGS. 17A and 17B, compliant foam 170 is used to centre the electrode as it is lifted off the skin. This configuration is similar to the arrangement illustrated in FIGS. 16A and 16B but, in the electrode up mode, a compliant foam 170 with tapered mating surfaces is used to centralise the electrode, thereby avoiding the need for lateral biasing cords. When in the electrode down mode, if the foam 170 contacts the electrode, this design may suffer from the trade-off problem mentioned above.

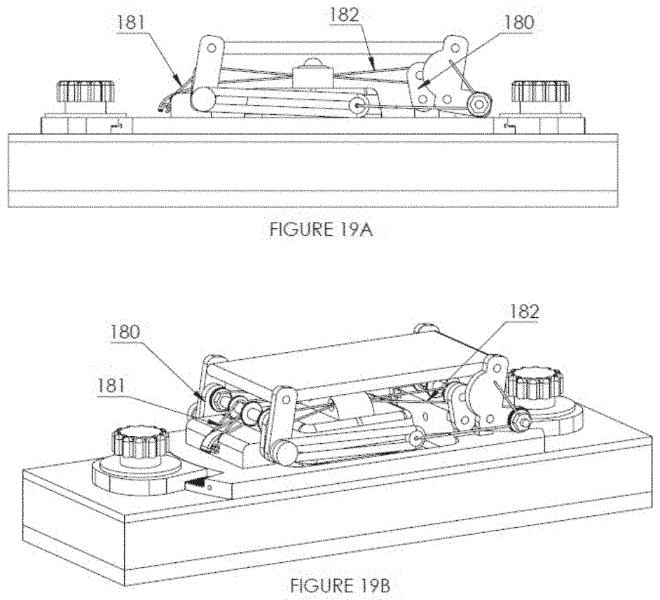

In FIGS. 18A to 21D, the main feature is the pair of sprung arms 180 that are pulled up by the elastic elements 181. These tension the stiff biasing cords 182 that return the electrode to its home position. When the plate 114 moves down to push the electrode against the skin, it also pushes the sprung arms down providing enough slack length in the stiff biasing cords to allow the electrode to move with the skin, not the socket.

FIG. 18A and FIG. 18B shows an arrangement with biasing arms 180 sprung using biasing cords 181 acting to tension stiff biasing cords 182 that hold the electrode in its home position.

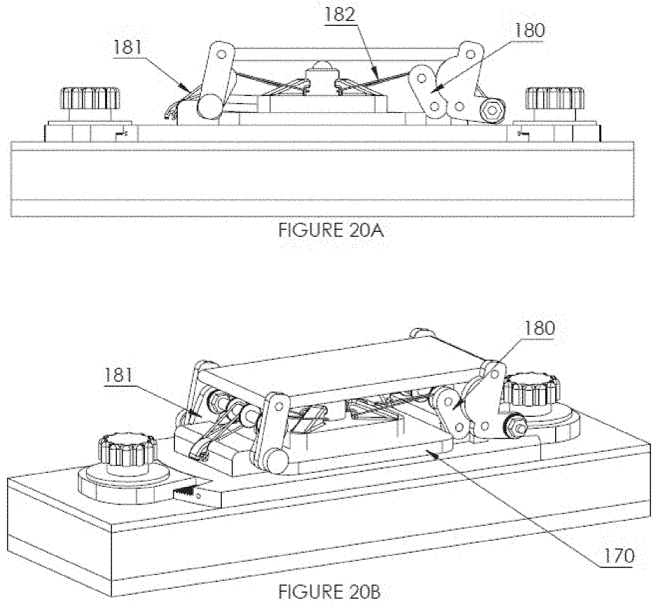

FIG. 19A and FIG. 19B shows a similar arrangement with sprung biasing arms acting to tension stiff biasing cords that hold the electrode in its home position.

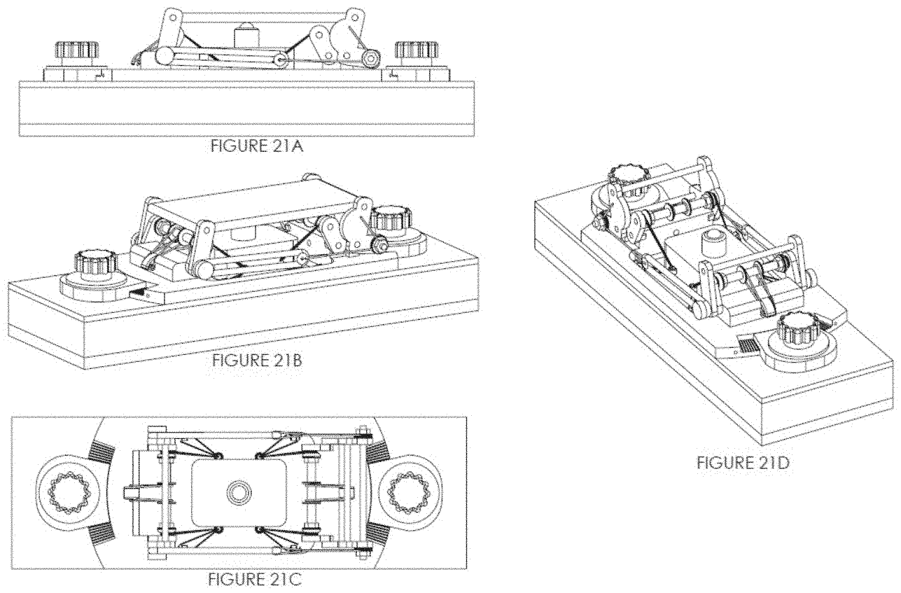

FIG. 20A and FIG. 20B shows bias arms with 4 high support bands return the electrode to its home position, and a compliant foam portion 170.

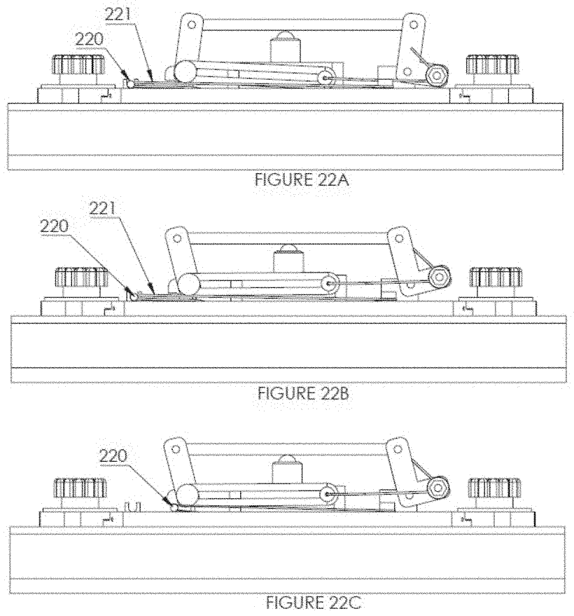

FIGS. 21A to 21D shows bias arms with corner cords high to low to return the electrode to its home position.

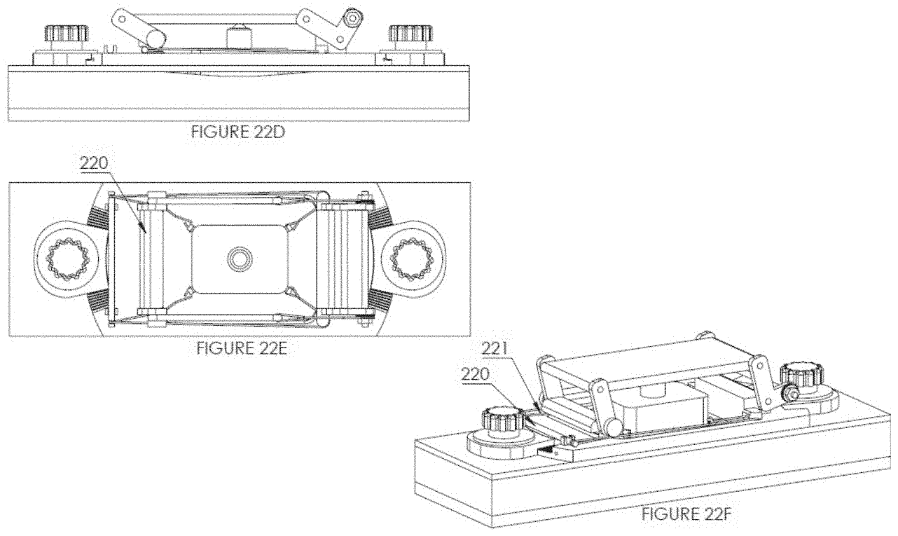

In the configuration illustrated in FIGS. 22A to 22F, the user operates the bias cords directly. A bar 220 means by which the user can independently control the biasing cords 221 is provided; tensioning them for electrode up mode and releasing them for electrode down mode. This is in addition to a means of lifting or lowering the electrode, here the bi-stable arrangement. Although two separate user controls are less preferred, this configuration could be used. One way to avoid two separate controls would be to allow the plate to remain in contact with the ball-unit (no bi-stable action); so that tensioning the bias cords lifts the electrode against the down-force of the pressure plate.

FIG. 22A shows the bi-stable lock engaged and the bias cord bar 220 in electrode up position. FIG. 22B shows the bi-stable lock disengaged, but the cord bar 220 still in electrode up position. In FIG. 22C, the cord bar 220 has been moved to electrode down position. FIG. 22D shows the electrode pushed against the skin.

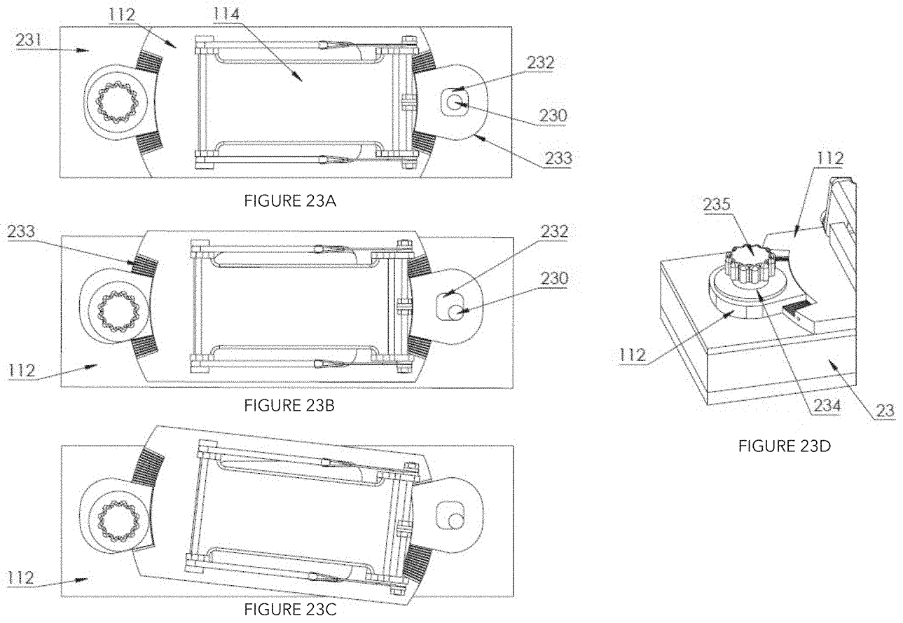

FIGS. 23A to 23D is a detailed isometric view of the engagement between the electrode housing and the mounting portions in the embodiment illustrated in FIG. 23A.

In this embodiment, studs 230 protrude from the socket 231, passing through oversize holes 232 in clamping tabs 233 which allow lateral movement of the electrode housing body 112 from the centred position. These holes 232 are in the clamping tabs 233 which clamp down the main electrode housing case 112 when the knurled knob 235 is tightened down onto an intermediate washer 234. The mating surfaces between tabs 233 and electrode housing case 112 are slightly serrated as illustrated to ensure high friction, and allow the electrode housing case 112 to move in an arc prior to being clamped. Overall, this configuration gives 5 mm of X and Y translation (dependent on the size of the holes 232 in the tabs 233), and 20 degrees of Z rotation (depending on the extent of the mating surfaces).

The one or more embodiments are described above by way of example only. Many variations are possible without departing from the scope of protection afforded by the appended claims.

1. An electrode assembly for a myoelectric prosthesis is provided, the electrode assembly comprising:
a. An electrode housing case having at least one adjustable mounting portion to mount the electrode housing case relative to a prosthesis socket and thereby mount the electrode assembly relative to a target area on the residual limb;
b. At least one electrode mounted relative to the electrode housing case by at least one biasing element to bias the at least one electrode into a home position relative to the electrode housing case and spaced from the target area;
c. A pressure transfer unit to transfer pressure to the at least one electrode in a direction toward the target area; and
d. A moveable pressure application structure operatively associated with the pressure transfer unit, the moveable pressure application structure mounted relative to the electrode housing case and relative to the pressure transfer unit to apply adjustable pressure to the at least one electrode toward the target area and moveable between at least one lowered position in which the at least one electrode is in contact with the target area and at least one raised condition in which the at least one electrode returns to the home position.

2. An electrode assembly as claimed in claim 1 wherein a shaped internal void is provided within the electrode housing case to contain but not prevent movement of the electrode within the electrode housing case.

3. An electrode assembly as claimed in claim 2 further comprising an electrode holder provided to hold the electrode relative to the electrode housing case and to abut the moveable pressure application structure when in the at least one lowered position.

4. An electrode assembly as claimed in claim 3 wherein the electrode holder is held relative to the electrode housing case using one or more resiliently deformable biasing bodies to allow movement of the electrode holder, and thereby the electrode, relative to the electrode housing case and socket so that the electrode is not dragged over the skin causing socket motion artefacts.

5. An electrode assembly as claimed in preceding claim 1 further comprising a pressure adjustment mechanism including an actuator mounted for user movement relative to the electrode housing case and a resiliently deformable adjustor mounted between the actuator and a portion of the moveable pressure application structure to adjust the pressure applied to the patient in the target area by the electrode.

6. An electrode assembly as claimed in claim 1 wherein the pressure transfer unit is provided at a lower end of the moveable pressure application structure, within the electrode housing case, the pressure transfer unit applying a force toward the patient, but otherwise allowing the electrode freedom of movement relative to the electrode housing case.

7. (canceled)

8. An electrode assembly as claimed in claim 2 wherein the pressure transfer unit is provided at an upper side of the electrode housing.

9. An electrode assembly as claimed in claim 1 wherein the pressure transfer unit is associated with an arcuate abutment portion.

10. An electrode assembly as claimed in claim 1 wherein the pressure transfer unit is associated with deformable unit to abut a portion of the movable pressure application structure.

11. An electrode assembly as claimed in claim 1 wherein the electrode housing case comprises at least one electrode mounting portion, provided adjacent to the opening through the electrode housing case and relative to which the at least one electrode mounted is mounted by the at least one biasing element to bias the at least one electrode into a home position.

12. An electrode assembly as claimed in claim 1 wherein the moveable pressure application structure comprises an abutment plate mounted relative to the electrode housing case using at least two arms.

13. (canceled)

14. (canceled)

15. (canceled)

16. An electrode assembly as claimed in claim 1 wherein the moveable pressure application structure is lockable in the up and/or down position.

17. An electrode assembly as claimed in claim 1 wherein the moveable pressure application structure is associated with at least one biasing assembly to bias the electrode into the electrode down position.

18. An electrode assembly as claimed in claim 17 wherein the at least one biasing assembly comprises at least one biasing element to provide a biasing force.

19. An electrode assembly as claimed in claim 18 wherein the at least one biasing element is selected to adjust the biasing force.

20. An electrode assembly as claimed in claim 17 when dependent from claim 15 wherein the at least one biasing assembly to bias the electrode into the electrode down position is associated with the crank arm.

21. An electrode assembly as claimed in claim 1 further comprising a threaded member provided relative to the electrode housing case to engage with the at least one mounting portion to releasably secure the electrode housing case in position once an optimum orientation is achieved.

22. An electrode assembly as claimed in claim 1 wherein the moveable pressure application structure is mounted relative to the electrode housing case, allowing movement of the pressure application structure toward and away from the patient and for it to be locked in the electrode up configuration.

23. An electrode assembly as claimed in claim 1 wherein the at least one biasing element is selected from the group including a spring, an elongate resilient member or elongate resilient loop.

24. A prosthesis comprising at least one electrode assembly according to claim 1.

25. (canceled)
